(19) United States
(12) Patent Application Publication
(10) Pub. No.: US 2005/0251176 A1
(43) Pub. Date: Nov. 10, 2005

(54) System for treating gastroesophageal reflux disease

(75) Inventors: Lee L. Swanstrom, Portland, OR (US); Vahid Saadat, Saratoga, CA (US); Eugene Chen, Carlsbad, CA (US); John A. Cox, Macungie, PA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: USGI Medical Inc., San Clemente, CA (US)

(21) Appl. No.: 11/102,571

(22) Filed: Apr. 7, 2005

Related U.S. Application Data
(63) Continuation-in-part of application No. 10/955,245, filed on Sep. 29, 2004.
Application No. 10/955,245 is a continuation-in-part of application No. 10/840,950, filed on May 7, 2004.

(51) Int. Cl. A61B 17/08

(52) U.S. Cl. 606/153

(57) ABSTRACT

A system for treating gastroesophageal reflux disease (GERD) is disclosed herein. A variety of tools, such as a shape-lockable endoscopic device, can be advanced trans-esophageally and into the stomach or through the stomach wall to access regions of the tissue in and around the gastroesophageal junction. Utilizing expandable tissue anchors, the angle of Hiss can be reconfigured by deploying the anchors within the esophagus and fundus and approximating the two. Alternatively, the esophagus can be lengthened by approximating tissue from within the stomach to follow the lesser curve of the stomach. Alternatively, one or more tissue folds can be formed within or adjacent to the GEJ to form a barrier to refluxing stomach contents.

### CROSS-REFERENCES TO RELATED APPLICATIONS

This is a continuation-in-part of U.S. patent application Ser. No. 10/955,245 (Attorney Docket No. 021496-003700US), filed Sep. 29, 2004, which is a continuation-in-part of U.S. patent application Ser. No. 10/840,950 (Attorney Docket No. 021496-000900US), filed May 7, 2004, each of which is incorporated herein by reference in its entirety.

### BACKGROUND OF THE INVENTION

Field of the Invention

The present invention relates to methods and apparatus for treating gastroesophageal reflux disease. More particularly, the present invention relates to methods and apparatus for endoluminally manipulating and/or securing tissue utilizing intra-gastric and/or extra-gastric approaches for the treatment of gastroesophageal reflux disease.

A number of techniques have been developed to treat various gastrointestinal disorders. One such example of a pervasive disorder is gastroesophageal reflux disease (GERD). Typical treatments usually involve modification of a patient's lifestyle including, e.g., dietary changes or changes in daily routine. Such lifestyle modifications may generally involve many factors such as maintaining an upright posture until a meal is fully digested or raising the head of the patient's bed to keep the patient's esophagus above the stomach. Other typical lifestyle modifications may also include avoiding physical exertion after a meal, or altering the time a patient eats and what types of food the patient can eat.

Aside from lifestyle modification, other typical treatments generally involve the use of prescription medication. Promotility drugs or H2 blockers, which reduce the amount of acid produced in the stomach, are typically only partially successful in alleviating GERD symptoms. Other drugs such as proton pump inhibitors (PPI) generally limit the amount of acid secretion in the stomach. PPIs typically allow for the rapid resolution of symptoms and for healing of the esophagus; however, patients may need to take medication for the rest of their lives as the underlying GERD condition remains.

Beyond pharmacological treatments, surgery is also utilized in patients for whom drugs are ineffective or for patients who do not wish to take drugs. However, surgical procedures may result in side effects such as difficulty in swallowing or the inability to belch or vomit. Furthermore, the sutures or staples that are often used in surgical procedures for GERD typically require extensive training by the clinician to achieve competent use, and may concentrate significant forces over a small surface area of the tissue, thereby potentially causing the suture or staple to tear through the tissue. The stomach, for instance, includes four tissue layers, where the mucosa layer is the inner-most tissue layer followed by connective tissue, the muscularis layer, and where the serosa layer is the outer-most tissue layer.

One problem with conventional surgical GERD treatments is that the anchors (or staples) should engage at least the muscularis tissue layer in order to provide a proper foundation. In other words, the mucosa and connective tissue layers typically are not strong enough to sustain the tensile loads imposed by normal movement of the stomach wall during ingestion and processing of food. In particular, these layers tend to stretch elastically rather than firmly hold the anchors (or staples) in position, and accordingly, the more rigid muscularis and/or serosa layer should ideally be engaged. This problem of capturing the muscularis or serosa layers becomes particularly acute where it is desired to place an anchor or other apparatus transesophageally rather than intra-operatively, since care must be taken in piercing the tissue wall not to inadvertently puncture adjacent tissue or organs.

One conventional method utilizes sewing devices to suture the tissue wall into folds. This procedure typically involves advancing a sewing instrument through the working channel of an endoscope and into or adjacent to the stomach and against the tissue. The contacted tissue is then typically drawn into the sewing instrument where one or more sutures or tags are implanted to hold the suctioned tissue in a folded condition known as a plication. Another method involves manually creating sutures for securing the plication.

One of the problems associated with these types of procedures is the time and number of intubations needed to perform the various procedures endoscopically. Another problem is the time required to complete a plication from the surrounding tissue with the body lumen. In the period of time that a patient is anesthetized, procedures such as for the treatment of GERD must be performed to completion. Accordingly, the placement and securement of the tissue plication should ideally be relatively quick and performed with a minimal level of confidence.

Moreover, when grasping or clamping onto or upon the layers of tissue with conventional anchors, sutures, staples, clips, etc., many of these devices are configured to be placed only after the tissue has been plicated and not during the actual plication procedure.

### BRIEF SUMMARY OF THE INVENTION

To affect various procedures for alleviating or eliminating GERD, various tools may be utilized endoluminally to engage, manipulate, and/or secure tissue in and around the stomach, gastroesophageal junction (GEJ), and/or esophagus. For example, a tool such as a shape-lockable endoscopic assembly may be advanced into a patient's stomach per-orally and through the esophagus. Such an endoscopic assembly may generally comprise an endoscopic device which may have a distal portion which may be articulated and steered to position its distal end anywhere within the stomach. Once desirably configured, the assembly may then be locked or rigidized to maintain its shape or configuration to allow for procedures to be performed on the tissue utilizing any number of tools delivered through the assembly. Shape-lockable assembly and its variations are described in further detail in U.S. patent application Ser. No. 10/734,562 filed Dec. 12, 2003, which is incorporated herein by reference in its entirety.

A distal steerable portion of the endoscopic body may be then articulated to an orientation, e.g., whereby distal portion facilitates engagement of tissue near and/or inferior to the patient's gastroesophageal junction. Accordingly, the distal steerable portion may comprise a number of steering features, as described in further detail in U.S. patent application Ser. No. 10/734,562, incorporated above. With the distal steerable portion disposed in a desired configuration or orientation, the endoscopic body may be reversibly shape-locked to a rigid state such that the endoscopic body maintains its position within the stomach. Various methods and apparatus for rigidizing endoscopic body 2 along its length are also described in further detail in U.S. patent application Ser. No. 10/734,562, incorporated above.

Utilizing this and other tools for grasping and manipulating tissue as well as anchor deployment assemblies, tissue anchors may be deployed within or against the tissue within the stomach, outside the stomach, within the esophagus, or elsewhere within the body in various configurations to effectively alleviate or eliminate the symptoms of GERD.

For example, one method for the treatment of GERD is to modify the angle of Hiss utilizing a trans-oral endoluminal intra-gastric or extra-gastric approach to configure the tissue in and around the gastroesophageal junction (GEJ). In this variation, expandable tissue anchors may be deployed, for instance, within the esophagus and within the stomach such that the anchors, when approximated towards one another and secured, draw the esophageal wall and fundus together. An intra-gastric approach may be performed by passing a needle assembly in an ante-grade or retro-grade fashion utilizing the shape-lockable endoscopic body.

In another variation, the tissue anchors may be deployed by advancing an endoscope trans-gastrically such that it passes within and through the stomach wall such that access is provided to the outer surfaces of the esophagus and stomach wall. Once the endoscope is extra-gastric, expandable tissue anchors may be deployed to modify the angle of Hiss.

In another method for treating GERD, tissue from within the stomach may be approximated to create a tissue ridge which roughly follows the lesser curvature of the stomach and effectively lengthens the esophagus. For instance, tissue regions along the anterior and posterior walls of the stomach inferior to the GEJ may be approximated and secured to one another. Detailed examples are described further in U.S. patent application Ser. Nos. 10/735,030 filed Dec. 12, 2003 and in 11/002,575 filed Dec. 1, 2004, each of which is incorporated herein by reference in its entirety.

In yet another method for the treatment of GERD, other procedures may be performed within or adjacent to the esophagus. For example, one or more tissue folds may be formed within or adjacent to the GEJ to help form a barrier to refluxing stomach contents. These tissue folds may be approximated towards one another about the periphery of the esophagus via suture to reduce the effective area of the GEJ. In securing the tissue folds, they may be interconnected via a length of suture, or the folds may be individually secured by anchor pairs deployed across each fold.

In yet another method for the treatment of GERD, the esophagus itself may be narrowed in the proximity of the GEJ by deploying one or more anchor pairs within or atop the diaphragm. The anchors may be tensioned in opposite directions and secured such that the resulting cross-section of the esophagus becomes narrowed.

### BRIEF DESCRIPTION OF THE DRAWINGS

FIG. 1 illustrates one example in which a shape-lockable endoscopic assembly may be advanced into a patient's stomach per-orally and through the esophagus with a tissue manipulation assembly advanced through a first lumen and a tissue engagement member advanced through a second lumen.

FIG. 2 illustrates a tissue manipulation assembly and examples of various tools which may be used in combination with the tissue manipulation assembly.

FIGS. 3A to 3D illustrate an example for performing an endoluminal tissue manipulation and securement procedure utilizing a tissue manipulation assembly in combination with a separate tissue grasping tool within, e.g., a patient's stomach.

FIG. 4A shows one variation where a single tissue fold may be secured between tissue anchors using the tissue manipulation assembly.

FIG. 4B shows another variation where two or more tissue folds may be secured between tissue anchors using the tissue manipulation assembly.

FIGS. 5A to 5E show an example where a needle assembly may be advanced through an opening along an endoscopic body and pierced through the esophageal wall and gastric wall to deploy expandable tissue anchors which may be approximated towards one another via a retroflexed portion of the endoscopic body.

FIG. 6 shows another variation similar to the assembly of FIGS. 5A to 5E where a needle assembly tube may be an integrated or separate tubular member attached to the outer surface of the endoscopic body.

FIGS. 7A and 7B show another variation in which a needle assembly may be advanced through the endoscopic body to pierce through tissue from within the stomach to within the esophagus.

FIG. 7C shows another example in which the needle assembly may be advanced in an ante-grade manner to pierce through tissue from within the esophagus to within the stomach.

FIGS. 8A to 8E show yet another example for deploying anchors via an extra-gastric approach.

FIGS. 9A and 9B show another example utilizing an extra-gastric approach where a hiatus opening in the diaphragm may be reduced via approximating anchors.

FIGS. 9C to 9E show an example for adhering tissue from a hiatal hernia and drawing it below the diaphragm within the peritoneal cavity.

FIGS. 9F to 9H show variations of an endoscopic body which may be used to temporarily adhere tissue thereto.

FIGS. 10A and 10B show side and partially open views of the stomach, respectively, where anchor pairs may be deployed for creating a pouch following the lesser curvature for the treatment of GERD.

FIG. 10C shows the stomach tissue configuration of FIG. 10A with an optional tissue fold created along the lesser curvature to further reduce an opening to the esophagus for treating GERD.

FIGS. 11A and 11B show side and partially open views of the stomach, respectively, where an anchor pair has been deployed and secured between the esophageal wall and gastric wall to modify the angle of Hiss.

FIG. 12A shows a side view of the stomach where multiple anchors have been deployed and secured between the esophageal wall and gastric wall.

FIG. 12B shows the reconfigured stomach of FIG. 12A with an optional tissue fold created along the lesser curvature to further reduce an opening to the esophagus for treating GERD.

FIG. 13 shows a stomach where one or more tissue folds may be formed within or adjacent to the gastroesophageal junction to form a barrier to refluxing stomach contents.

FIGS. 14A and 14B show top and side views, respectively, of one example of the esophagus with one or more tissue folds formed in or around the GEJ utilizing tissue anchors connected via suture through the tissue folds.

FIGS. 15A and 15B show top and side views, respectively, of another example where the esophagus has at least two tissue folds approximated towards one another with first and second anchors.

FIGS. 16A and 16B show top and side views, respectively, of another example where the anchors are positioned either below the tissue folds or atop the tissue folds and joined via the suture.

FIGS. 17A and 17B show top and side views, respectively, of another example where tissue folds are formed adjacent to one another with anchor pairs securing their respective tissue folds such that each individual fold has an anchor atop and below the fold for maintaining the tissue fold configuration.

FIG. 18 shows an alternative variation where a tissue ridge may be formed below or inferior to the gastroesophageal junction such that the ridge extends from the lesser curvature and into the stomach cavity.

FIG. 19 shows another alternative where the tissue ridge may also be formed adjacent to the gastroesophageal junction such that it extends into the stomach and towards the greater curvature of the stomach.

FIGS. 20A and 20B show side and top views of the stomach, respectively, where a distal anchor may be first deployed against the esophageal wall with the suture pierced through the esophagus and fundus and then pierced back through the fundus and esophagus forming a U-stitch through the tissue layers.

FIGS. 21A and 21B show side and top views of the stomach, respectively, in another example where a U-stitch may be formed within the tissue but with anchors having been deployed within the stomach and with the suture passed through the fundus, into the esophagus, and then back through the esophagus and through the fundus.

FIGS. 22A and 22B show side and top views of the stomach, respectively, where tissue anchors may be deployed within the esophagus and on the outer surface of the stomach along the greater curvature with suture extending between the two anchors.

FIG. 23A shows a side view of the stomach and diaphragm in another variation where the esophagus may be attached to the diaphragm for narrowing the cross-sectional area of the esophagus for treating GERD.

FIG. 23B shows a top view of the esophagus from FIG. 23A showing the resulting reduced cross-sectional area of the esophagus.

### DETAILED DESCRIPTION OF THE INVENTION

In manipulating tissue or creating tissue folds, a having a distal end effector may be advanced endoluminally, e.g., transorally, transgastrically, etc., into the patient's body, e.g., the stomach. The tissue may be engaged or grasped and the engaged tissue may be manipulated by a surgeon or practitioner from outside the patient's body. Examples of creating and forming tissue plications may be seen in further detail in U.S. patent application Ser. No. 10/955,245 filed Sep. 29, 2004, which has been incorporated herein by reference above, as well as U.S. patent application Ser. No. 10/735,030 filed Dec. 12, 2003, which is incorporated herein by reference in its entirety.

In engaging, manipulating, and/or securing the tissue, various procedures may be accomplished. For instance, tissue securement devices may be delivered and positioned via an endoscopic apparatus for contacting a tissue wall of the gastrointestinal lumen, creating one or more tissue folds, and deploying one or more tissue anchors through the tissue fold(s). The tissue anchor(s) may be disposed through the muscularis and/or serosa layers of the gastrointestinal lumen.

One such procedure which may be accomplished is for the treatment of gastroesophageal reflux disease (GERD). To affect various procedures for alleviating or eliminating GERD, various tools may be utilized endoluminally to engage, manipulate, and/or secure tissue in and around the stomach, gastroesophageal junction (GEJ), and/or esophagus.

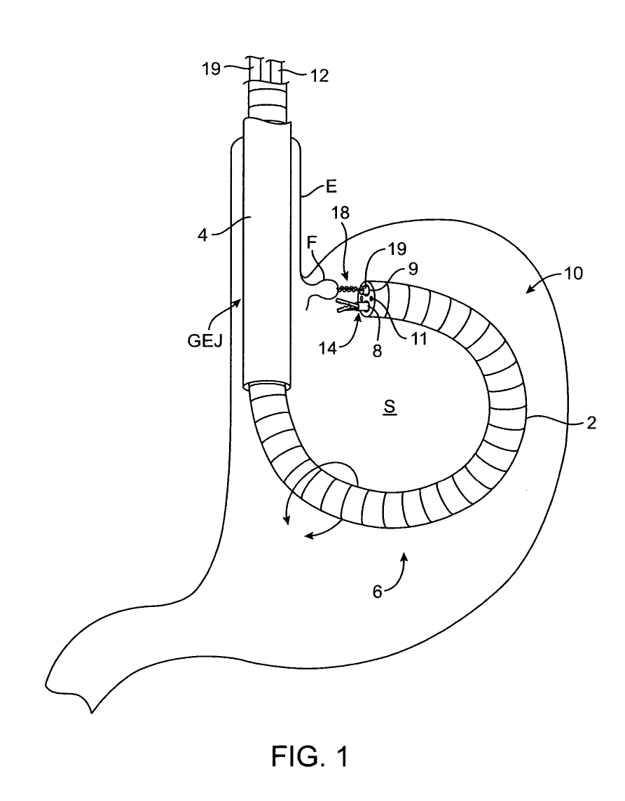

As illustrated in FIG. 1, one such example of a tool is shown in which a shape-lockable endoscopic assembly 10 may be advanced into a patient's stomach S per-orally and through the esophagus E. Such an endoscopic assembly 10 may generally comprise an endoscopic device which may have a distal portion which may be articulated and steered to position its distal end anywhere within the stomach S. Once desirably configured, assembly 10 may then be locked or rigidized to maintain its shape or configuration to allow for procedures to be performed on the tissue utilizing any number of tools delivered through the assembly 10. Shape-lockable assembly 10 and its variations are described in further detail in U.S. patent application Ser. No. 10/734,562 filed Dec. 12, 2003, which is incorporated herein by reference in its entirety.

Shape-lockable assembly 10 may be generally comprised of shape-lockable endoscopic body 2 having an articulatable distal portion 6. The endoscopic body 2 may define at least first and second lumens 8, 9, respectively, through the endoscopic body 2 through which one or more tools may be deployed into the stomach S. Additional lumens may be provided through shape-lockable endoscopic body 2, such as a visualization lumen 11, through which an endoscope may be positioned to provide visualization of the region of tissue. Alternatively, an imager such as a CCD imager or optical fibers may be provided in lumen 11 to provide visualization. An optional thin wall sheath 4 may be disposed through the patient's mouth, esophagus E, and possibly past the gastroesophageal junction GEJ into the stomach S. Shape-lockable body 2 may be advanced through esophagus E (and through sheath 4, if utilized) and into stomach S while disposed in a flexible state.

Distal steerable portion 6 of endoscopic body 2 may be then articulated to an orientation, e.g., whereby distal portion 6 facilitates engagement of tissue near and/or inferior to the patient's gastroesophageal junction GEJ. Accordingly, distal steerable portion 6 may comprise a number of steering features, as described in further detail in U.S. patent application Ser. No. 10/734,562, incorporated above. With distal steerable portion 6 disposed in a desired configuration or orientation, endoscopic body 2 may be reversibly shape-locked to a rigid state such that the endoscopic body 2 maintains its position within the stomach S. Various methods and apparatus for rigidizing endoscopic body 2 along its length are also described in further detail in U.S. patent application Ser. No. 10/734,562, incorporated above.

FIG. 1 shows tissue manipulation assembly 14 having been advanced through first lumen 8 and a tissue engagement member 18 positioned upon flexible shaft 19 advanced through second lumen 9. As the tissue wall of a body lumen, such as the stomach, typically comprises an inner mucosal layer, connective tissue, the muscularis layer and the serosa layer. To obtain a durable purchase, e.g., in performing a procedure for alleviating GERD, tissue engagement member 18 may be advanced into contact with the tissue and preferably engages the tissue F such that when the tissue engagement member 18 is pulled proximally to draw the engaged tissue F between the jaw members of tissue manipulation assembly 14, at least the muscularis tissue layer and the serosa layer is drawn into tissue manipulation assembly 14, as described in further detail below.

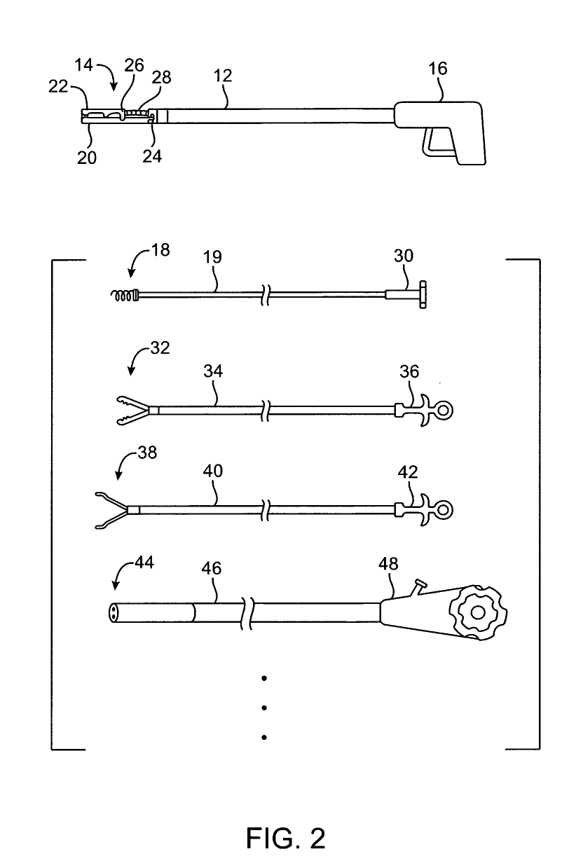

As tissue manipulation assembly 14 may be utilized to grasp and secure the engaged tissue, any number of tools may be utilized with tissue manipulation assembly 14, e.g., through shape-lockable endoscopic body 2, to engage and manipulate the tissue of interest relative to tissue manipulation assembly 14. FIG. 2 illustrates tissue manipulation assembly 14 upon flexible body 12 with handle 16 and examples of various tools which may be used in combination with tissue manipulation assembly 14.

As shown in FIG. 2, tissue manipulation assembly 14 generally comprises a flexible catheter or tubular body 12 which may be configured to be sufficiently flexible for advancement into a body lumen, e.g., transorally, percutaneously, laparoscopically, etc. Tubular body 12 may be configured to be torqueable through various methods, e.g., utilizing a braided tubular construction, such that when handle 16 is manipulated and/or rotated by a practitioner from outside the patient's body, the longitudinal and/or torquing force is transmitted along body 12 such that the distal end of body 12 is advanced, withdrawn, or rotated in a corresponding manner.

Tissue manipulation assembly 14 is located at the distal end of tubular body 12 and is generally used to contact and form tissue folds, as mentioned above, and is connected to the distal end of tubular body 12 via a pivotable coupling 24. Lower jaw member 20 extends distally from the pivotable coupling 24 and upper jaw member 22, in this example, may be pivotably coupled to lower jaw member 20 via jaw pivot 26. The location of jaw pivot 26 may be positioned at various locations along lower jaw 20 depending upon a number of factors, e.g., the desired size of the “bite” or opening for accepting tissue between the jaw members, the amount of closing force between the jaw members, etc. One or both jaw members 20, 22 may also have a number of protrusions, projections, grasping teeth, textured surfaces, etc., on the surface or surfaces of the jaw members 20, 22 facing one another to facilitate the adherence of tissue between the jaw members 20, 22.

Tissue manipulation assembly 14 is described in further detail in U.S. patent application Ser. No. 11/070,863 (Attorney Docket No. 021496-003760US), filed Mar. 1, 2005, which is incorporated herein by reference in its entirety. Other tissue manipulation and engagement tools which may also be utilized may be seen in U.S. patent application Ser. No. 10/955,245 filed Sep. 29, 2004, which is also incorporated herein by reference in its entirety.

One example of a tool utilizable in combination with tissue manipulation assembly 14 is shown in tissue engagement member 18 as a tissue piercing helix or corkscrew structure upon flexible shaft 19 (as shown in FIG. 1). Tissue engagement member 18 may be rotated about its longitudinal axis to engage the tissue of interest by rotating handle 30 located on the proximal end of flexible shaft 19. Alternatively, a tool having aggressive tissue graspers 32 positioned upon flexible shaft 34 and articulatable via handle 36 may be utilized in combination with tissue manipulation assembly 14. Another alternative tool may be tissue graspers 38 positioned upon flexible shaft 40 and articulatable via handle 42. Tissue graspers 38 may have atraumatic grasping surfaces. In yet another alternative, an endoscope 46 having optical fibers or imager 44 may be utilized for providing visualization. Endoscope 46 may be articulated via handle 48 at its proximal end.

The examples of the various tools as shown and described are intended merely to be illustrative of the range of tools which may be usable with assembly 14 and are not intended to be limiting in any manner. Any number of other tools may be accordingly utilized and are intended to be within the scope of this disclosure.

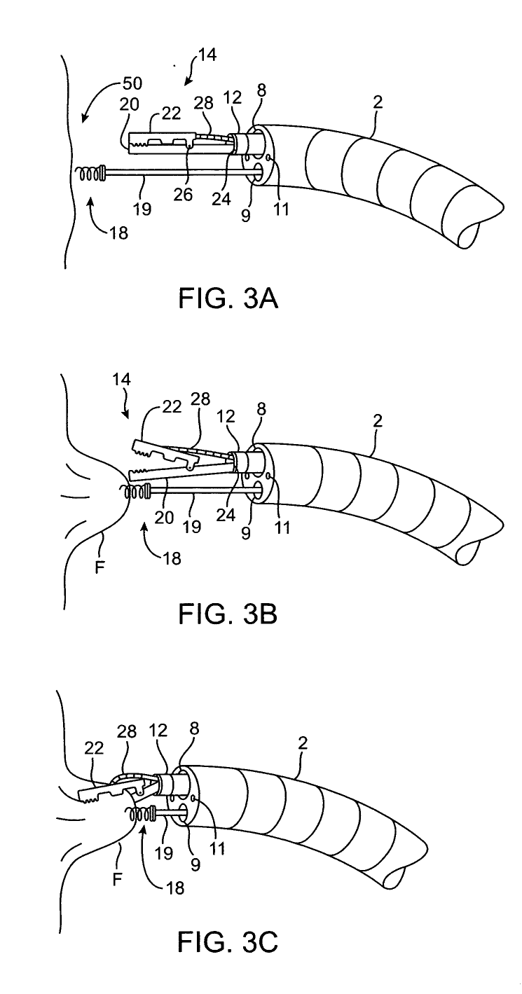

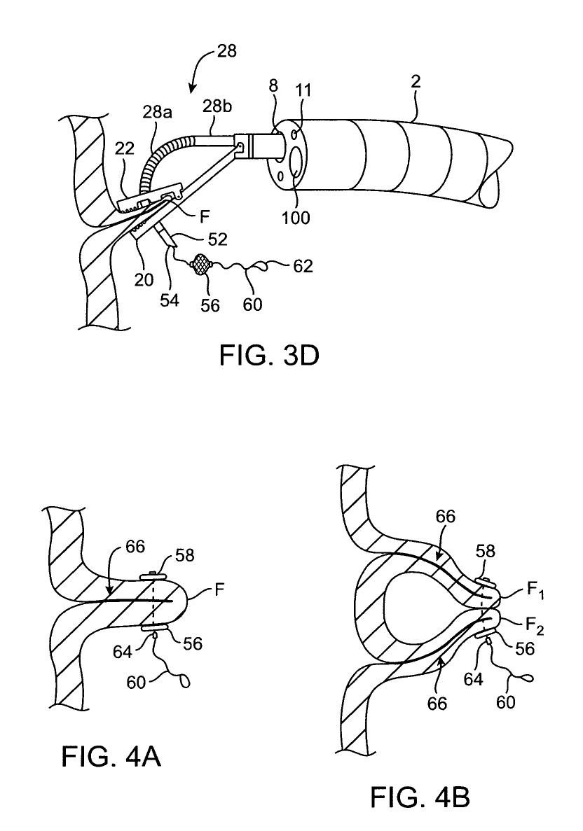

An example of performing an endoluminal tissue manipulation and securement procedure utilizing tissue manipulation assembly 14 in combination with a separate tissue grasping tool within, e.g., a patient's stomach, is illustrated in FIGS. 3A to 3D. As shown in FIG. 3A, once shape-lockable endoscopic body 2 has been introduced into the patient, e.g., trans-orally, trans-anally, percutaneously, etc., and desirably positioned relative to a tissue region of interest 50, endoscopic body 2 may be rigidized to maintain its configuration within the patient body. Alternatively, it may be left in a flexible state during the procedure.

The tissue region of interest 50 as well as the procedure may be visualized through visualization lumen 11 or a separate imager, as described above. In either case, tissue manipulation assembly 14 and tissue engagement member 18 may be advanced distally out from endoscopic body 2 through their respective lumens 8, 9. Tissue engagement member 18 may be advanced into contact against the tissue surface, as shown in FIG. 3A, and then rotated via its proximal handle until the tissue is engaged. The engaged tissue F may be pulled proximally relative to endoscopic body 2 and tissue manipulation assembly 14 may be actuated via its proximally located handle into an open expanded jaw configuration for receiving the engaged tissue F, as shown in FIG. 3B.

Alternatively, once the tissue F has been engaged, tissue manipulation assembly 14 may be advanced distally in its open configuration onto the engaged tissue. In yet another variation, tissue engagement member 18 may be omitted entirely and tissue manipulation assembly 14 may be utilized alone to grasp onto the tissue region of interest 50. In yet another alternative, a second tissue manipulation assembly may be used in combination with tissue manipulation assembly 14.

Turning back to FIG. 3B, tissue manipulation assembly 14 may be articulated to receive the engaged tissue F. As shown in FIG. 3C, once engaged tissue F is positioned between jaw members 20, 22, the launch tube 28 may be urged proximally to actuate upper jaw member 22 to grasp or clamp upon the tissue F. Tissue engagement member 18 may be retracted from the tissue F or it may be left within the tissue while tissue manipulation assembly engages and secures the tissue F.

FIG. 3D shows a partial cross-sectional view of the tissue F while engaged to tissue manipulation assembly 14. Tissue engagement member 18 has been omitted from this view only for the sake of clarity. As mentioned above, member 18 may be left remaining in the tissue F, disengaged from tissue F, or disengaged and removed entirely from endoscopic body 2, if so desired, and another tool may be advanced through lumen 9 to facilitate the procedure. Once the tissue has been pulled or manipulated between jaw members 20, 22, launch tube 28 may be pushed distally to actuate the jaw members 20, 22 into a closed, grasping configuration for engagement with the tissue. Launch tube 28 may further define a flexible portion 28a distally of a rigid portion 28b.

Once jaw members 20, 22 have been actuated to clamp or grasp upon tissue F by the launch tube 28, the launch tube 28 may be automatically positioned into its anchor deployment configuration. The needle assembly may then be urged via manipulation from its proximal end at handle 16 through the launch tube 28 to pierce preferably through a dual serosa layer through engaged tissue F and past lower jaw member 20. As described above, the engaged tissue F positioned between the jaw members 20, 22 is desirably engaged such that the needle body 52, when urged through the tissue F, is disposed through the muscularis and/or serosa layers of the engaged tissue F. Once needle body 52 has passed through tissue F, one or more expandable tissue anchors may be ejected from needle body 52 through needle opening 54.

Because needle body 52 may penetrate the tissue wall twice, it exits within the body lumen if utilized within, e.g., the stomach, thus reducing the potential for injury to surrounding organs. As described above, needle body 52 may define needle lumen or opening 54 through which expandable an anchor, e.g., distal anchor 56 and/or proximal anchor 58, may be situated during deployment and positioning of the assembly. A single suture or flexible element 60 (or multiple suture elements) may connect distal anchor 56 and proximal anchor 58 to one another and end in terminal loop 62. For instance, element 60 may comprise various materials such as monofilament, multifilament, or any other conventional suture material, elastic or elastomeric materials, e.g., rubber, etc.

Once distal anchor 56 has been ejected, needle body 52 may be urged proximally back through tissue F, where proximal anchor 58 may then be ejected from needle body 52 with suture 60 still connecting the two anchors 56, 58 through tissue F. Alternatively, tissue manipulation assembly 14, with suture 60 still depending therefrom, may be disengaged from tissue F and the procedure may be repeated at a second region of tissue where proximal anchor 58 may then be ejected.

FIG. 4A shows one variation where a single fold F may be secured between proximal anchor 58 and distal anchor 56. With both anchors 56, 58 disposed externally of the launch tube 28 and suture 60 connecting the two, the anchors 56, 58 may be urged into contact against tissue F. As the anchors are urged against tissue fold F, distal anchor 56 or a portion of suture 60 may be configured to provide any number of directionally translatable locking mechanisms 64 which provide for movement of an anchor along suture 60 in a first direction and preferably locks, inhibits, or prevents the reverse movement of the anchor back along suture 60.

FIG. 4B shows another variation where at least two folds F₁ and F₂ may be secured between proximal anchor 58 and distal anchor 56. After the anchors have been ejected from needle body 52, the anchors may be approximated towards one another over suture 60 thus bringing folds F₁ and F₂ towards one another. Although a single tissue fold and a dual fold are shown in these examples, any number of folds or tissue ridges may be created using the tools disclosed herein. Moreover, these examples are merely intended to be illustrative and not limiting in any way. In either case, it may be generally desirable to form the tissue folds such that serosa-to-serosa contact 66 occurs between the layers of secured tissue, although this may not be necessary.

Various examples of cinching devices and methods which may be utilized with the tools and devices herein are described in further detail in U.S. patent application Ser. No. 10/840,950 filed May 7, 2004, which is incorporated herein in its entirety.

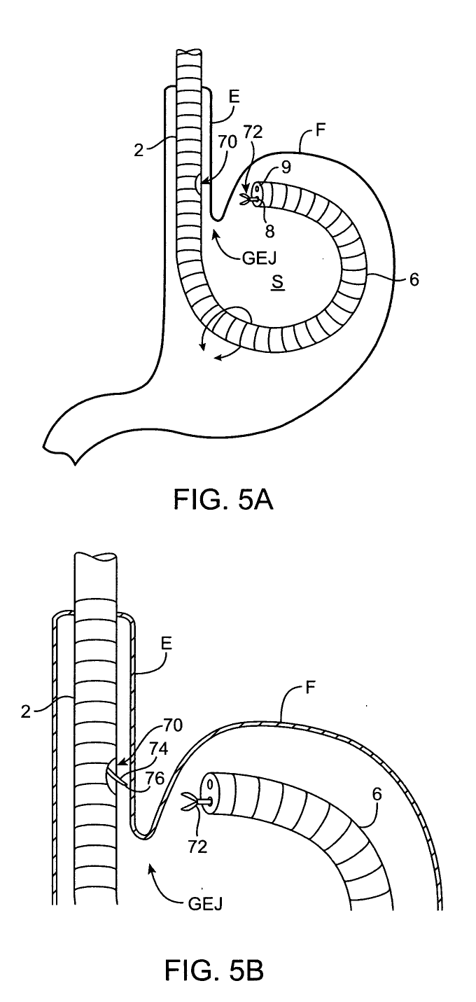

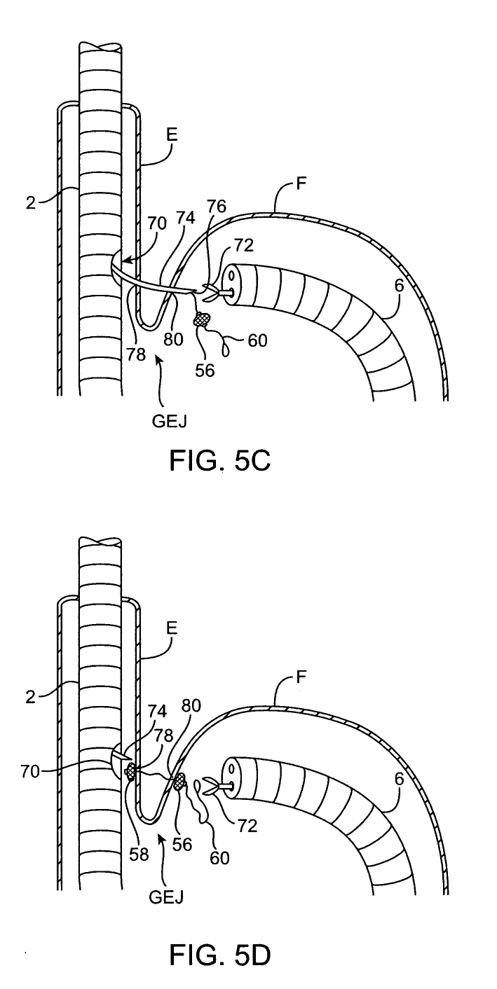

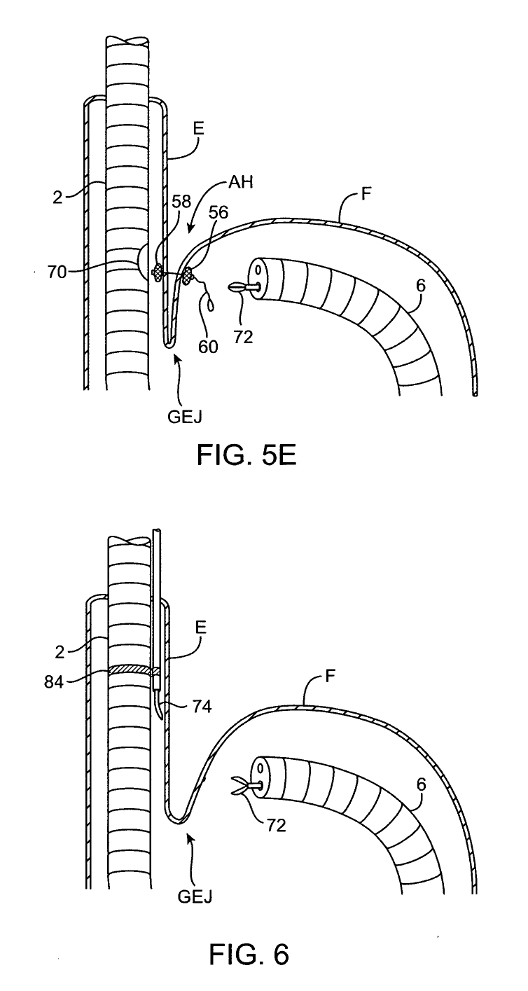

Another example of a tool for manipulating tissue and/or deploying tissue anchors in and around, e.g., the stomach and/or esophagus, which may be utilized for the treatment of GERD is shown in FIGS. 5A to 5E. In this example, endoscopic body 2, as described above, may define a skived region or opening 70 along a length of the body 2. With endoscopic body 2 advanced, e.g., through esophagus E, distal steerable portion 6 may be articulated into a retroflexed configuration and positioned within the fundus F of stomach S, as shown in FIG. 5A. Distal steerable portion 6 may be articulated such that first and second lumens 8, 9, respectively, are positioned adjacent to the tissue wall within stomach S superior to the GEJ and pointing towards the general vicinity of opening 70, which is positioned within esophagus E also superior to the GEJ.

Once desirably positioned, a needle assembly 74 may be advanced through endoscopic body 2 until it exits from opening 70. The skive or opening 70 may have a ramped portion (not shown) to direct the needle assembly 74 out of opening 70, as shown in FIG. 5B. The needle assembly 74 may contain one or more soft tissue anchors slidably positioned within for deployment through needle opening 76 for securing portions of the tissue. Graspers 72 may be likewise advanced through lumen 8 prior to, after, or simultaneously with needle assembly 74 being advanced through opening 70. As needle assembly 74 is slowly advanced, it may pierce through the esophageal tissue wall forming opening 78 and through the gastric tissue wall forming an opening 78 into the fundus F, as shown in FIG. 5C. The location of tissue opening 78 may be varied along the esophageal wall proximally of or adjacent to GEJ. Likewise, the corresponding tissue opening 80 formed in the gastric wall and leading into fundus F may also be varied proximally of or adjacent to GEJ.

As shown in FIG. 5D, once needle opening 76 is positioned within stomach S, suture 60 and distal anchor 56 may be deployed into stomach S. With suture 60 trailing, needle assembly 74 may be withdrawn proximally through gastric tissue opening 80 and esophageal tissue opening 78 with distal anchor 56 remaining within stomach S. Once needle assembly 74 has been withdrawn back into opening 70 within esophagus E, proximal anchor 58 may be deployed from needle assembly 74. Suture 60 may be grasped by grasper 72 and pulled proximally while advancing a cinching mechanism (not shown) over suture 60 such that distal and proximal anchors 56, 58 are drawn towards one another and expanded against their respective tissue surfaces, as shown in FIG. 5E. As the anchors 56, 58 are drawn towards one another along suture 60, the angle of Hiss AH becomes modified as the outer surfaces of the fundus and the esophageal tissue are brought towards one another and secured. As the anchors 56, 58 are pressed against the respective tissue surfaces, the anchors are expanded from their low-profile configuration to an expanded and deployed configuration which prevents the anchors from being pulled through the tissue and ensures securement of the tissue layers against one another.

Soft tissue anchors, anchor variations, and methods for deploying and securing the anchors may be seen in further detail in U.S. patent application Ser. No. 10/869,472 filed Jun. 14, 2004. Additional details may also be seen on various cinching tools for drawing the anchors towards one another to secure the underlying tissue in U.S. patent application Ser. No. 10/954,665 filed Sep. 29, 2004. Each patent application is incorporated herein by reference in its entirety.

In an alternative approach, rather than delivering needle assembly 74 through endoscopic body 2 and out through opening 70, FIG. 6 shows an approach in which a needle assembly tube 82 positioned adjacently along endoscopic body 2 may be positioned over body 2 such that a needle assembly 74 advanced therethrough may be positioned to pierce through the esophageal tissue wall. Needle assembly tube 82 may be an integrated tubular lumen through endoscopic body 2, or it may comprise a separate tubular member attached to the outer surface of body 2 via one or more bands or straps 84 over a length of assembly tube 82. In this variation, once endoscopic body 2 has been desirably positioned within esophagus E and stomach S, needle assembly 74 may be advanced through assembly tube 82 such that needle assembly 74 pierces through esophagus E and the gastric wall, as described above, such that the anchors may be deployed and secured for altering the angle of Hiss.

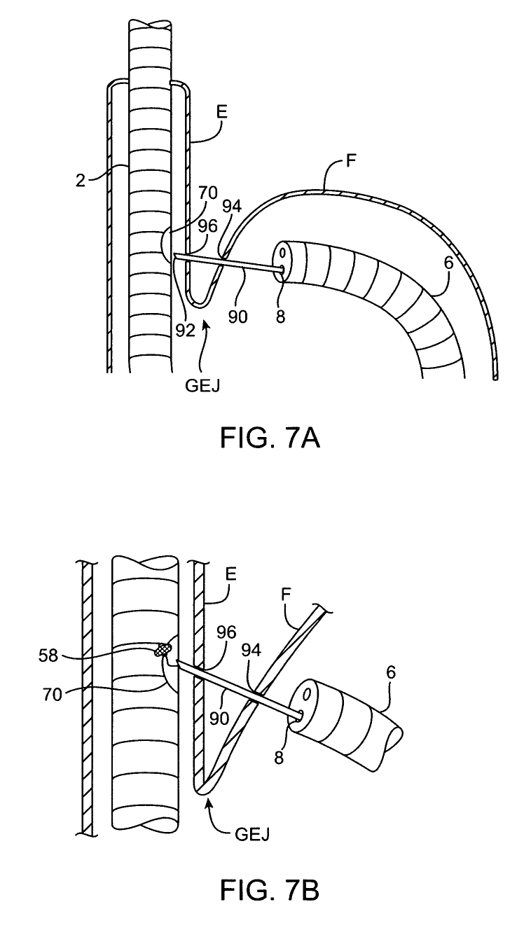

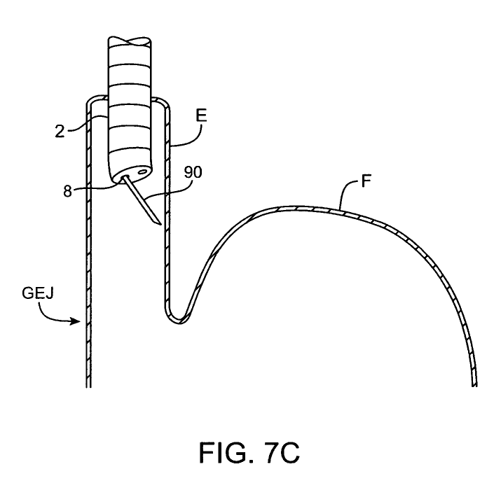

In yet another example shown in FIGS. 7A and 7B, a needle assembly 90 having a needle opening 92 may be advanced through the length of endoscopic body 2 via a working lumen such as first lumen 8. Endoscopic body 2 may be articulated within stomach such that its distal end is directed towards the endoscopic tissue wall, as described above. Needle assembly 90 may then be advanced distally such that it pierces through the gastric wall forming tissue opening 94 and through the esophageal wall forming tissue opening 96, as shown in FIG. 7A. Tissue openings 94, 96 may be positioned superior to the GEJ, as described above; however, the locations may be altered depending upon the desired results.

After needle assembly 90 has been advanced through the tissue and into esophagus E, distal anchor 58 may be deployed from needle assembly 90 for expansion against the esophageal tissue. As shown in FIG. 7B, opening 70 along endoscopic body 2 may be optionally provided to allow an opening or space for deploying distal anchor 58 within esophagus E if little or no space remains, depending upon the patient's anatomy, between endoscopic body 2 and the wall of esophagus E. Once distal anchor 58 is deployed, needle assembly 90 may be withdrawn proximally back into stomach S, where the proximal anchor may then be deployed and the two anchors may be drawn towards one another, as described above.

FIG. 7C shows yet another example in which the endoscopic body 2 may be advanced partially into the esophagus E until its distal end is at a location superior to the GEJ within the esophagus E. Endoscopic body 2 may then be shape-locked to maintain its configuration while needle assembly 90 is advanced through first lumen 8 to pierce through the tissue of the esophagus E and fundus F, where a tissue anchor may be deployed. The needle assembly 90 may then be withdrawn proximally back into the esophagus E where the second tissue anchor may be deployed. The two anchors may then be approximated towards one another, as above.

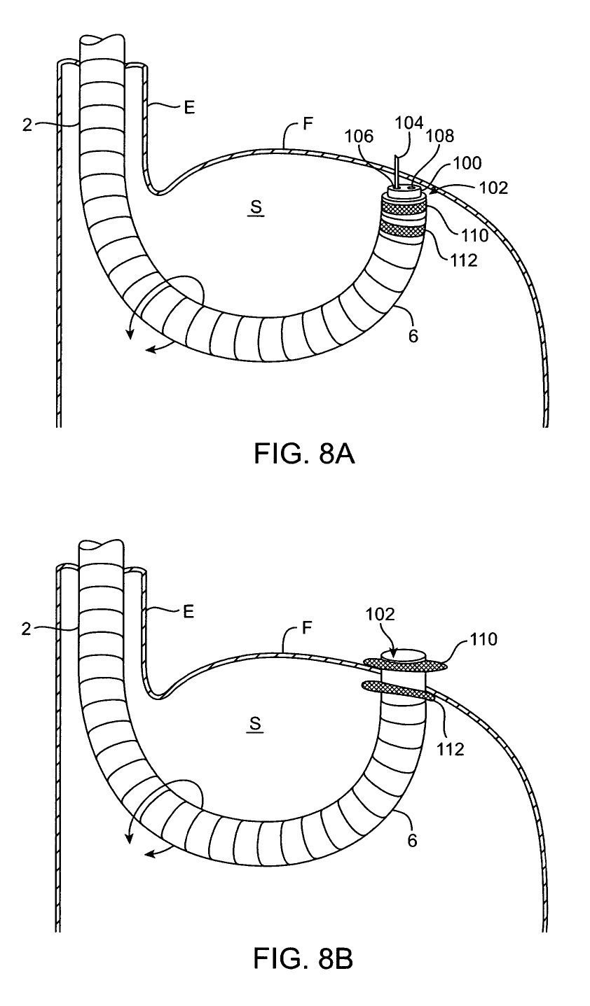

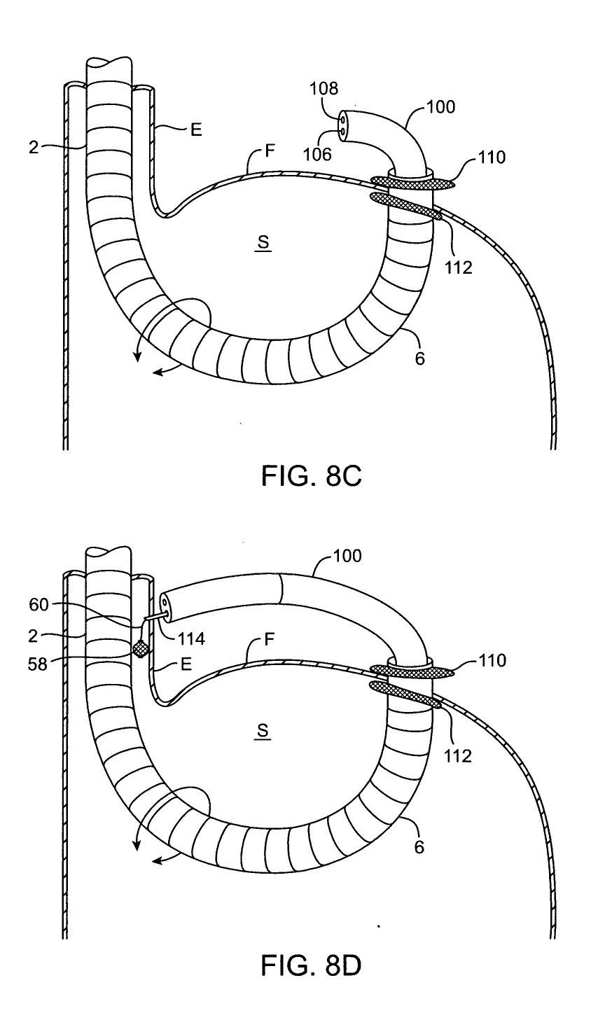

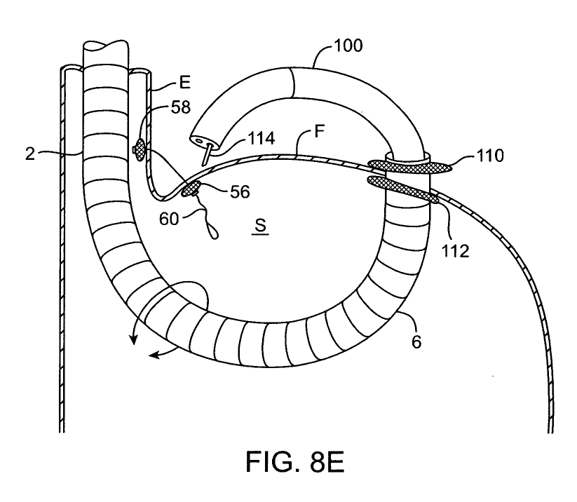

Aside from endoluminal approaches towards and around the GEJ, extra-gastric approaches may also be utilized for treating GERD. For instance, one example is shown in FIG. 8A in which endoscopic body 2 may be advanced through esophagus E and into stomach S, as described above. As described above, endoscopic body 2 may comprise a shape-lockable body configured to be advanced through the body in a flexible state and then rigidized along its length, or at least a portion of its length, to retain a configuration and to provide a stable platform from which to perform any number of procedures. In this example, endoscopic body 2 may include one or more expandable members 110, 112, which may comprise any number of expandable structures (e.g., balloons, mesh structures, scaffolding, etc.), which are spaced apart from one another.

After endoscopic body 2 has been advanced and articulated to a tissue region, e.g., along the fundus F of the stomach S, endoscopic body 2 may be optionally rigidized to maintain its configuration, as shown in the FIG. 8A. An endoscope 100 may be advanced through a lumen 102 of endoscopic body 2 and a cutting and/or dilating tool, e.g., an obturator or needle knife 104 (as shown), may be advanced through a first lumen 106 through endoscope 100. A second lumen 108 may be utilized as a visualization lumen. Needle knife 104, in this example, may be advanced to cut an opening or dilate an opening through the gastric tissue to allow for advancement of the distal end of endoscopic body 2 through the newly created opening. Once expandable member 110 has been advanced past the gastric opening in the fundus wall, expandable members 110, 112 may be expanded to sandwich the gastric tissue therebetween and anchor a position of the endoscopic body 2 relative to the tissue, as shown in FIG. 8B. Alternatively, the distal end of endoscopic body 2 may be anchored to the gastric opening through a number of other methods rather than advancing the device 2 itself through the opening.

Once the endoscopic device has been anchored to the gastric wall, endoscope 100 may be advanced through endoscopic body 2 and into the peritoneal cavity, as shown in FIG. 8C, or the thoracic cavity, if desired. In this example, endoscope 100 may be articulated towards a region along the esophagus E superior to the GEJ, as shown in FIG. 8D, and needle assembly 114 may be urged to project distally from endoscope 100 until it pierces through a region along esophagus E. Once needle assembly 114 is at least partially within esophagus E, distal anchor 58 may be deployed. Needle assembly 114 may then be retracted proximally from esophagus E with suture 60 trailing from needle assembly 114. Endoscope 100 may then be repositioned to a location along the outer serosal surface of stomach S to a location along the fundus F where needle assembly 114 may again be advanced until it pierces through the gastric wall.

Once back inside the stomach S, proximal anchor 56 may be deployed, as shown in FIG. 8E. After the anchors 56, 58 have been deployed within esophagus E and stomach S, endoscope 100 and endoscopic body 2 may be retracted back into the stomach S, where a grasping and/or cinching tool may be utilized to draw the anchors 56, 58 towards one another such that the angle of Hiss is reconfigured.

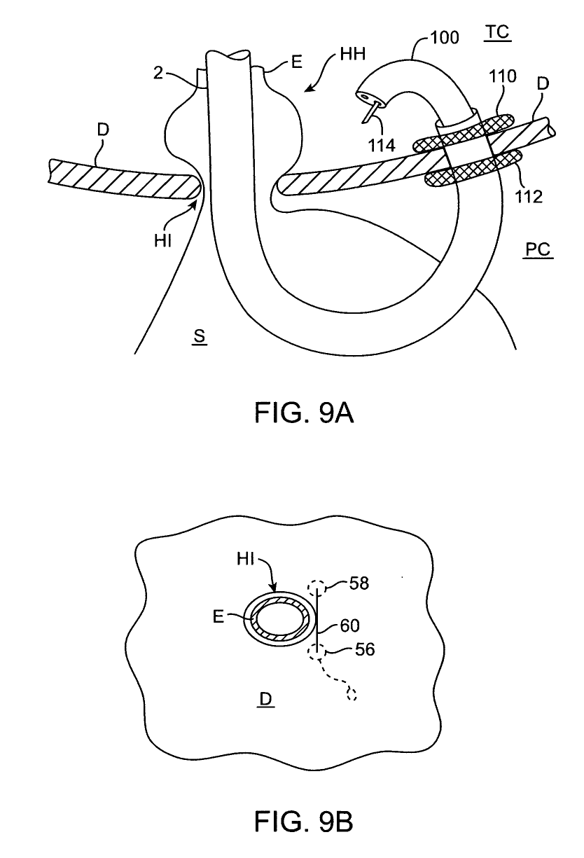

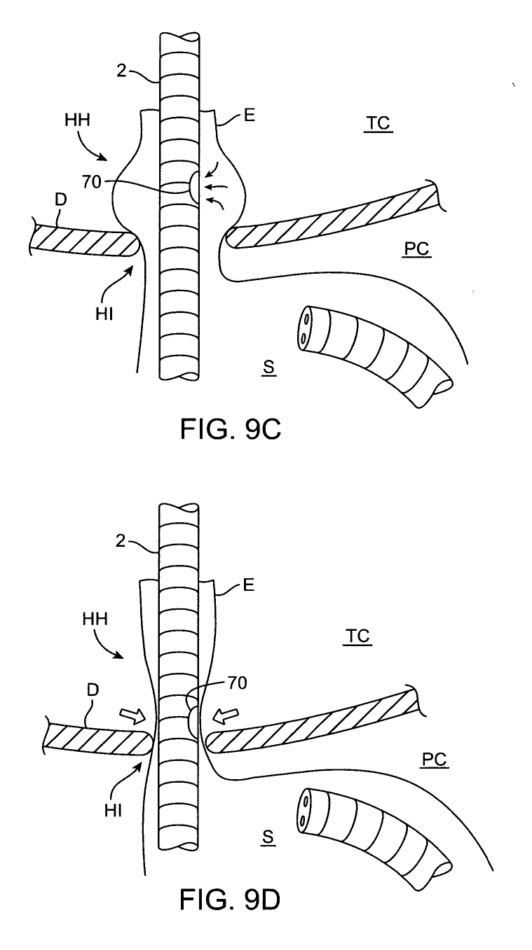

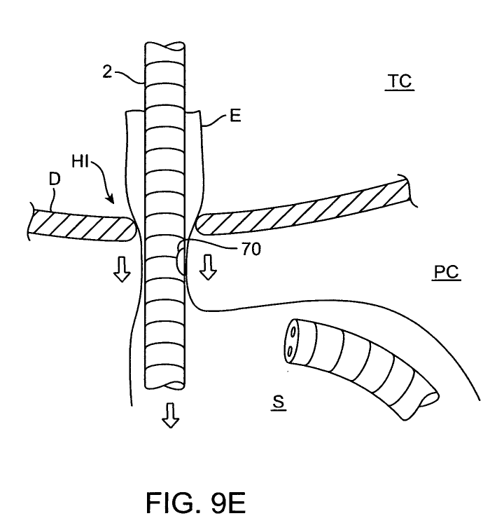

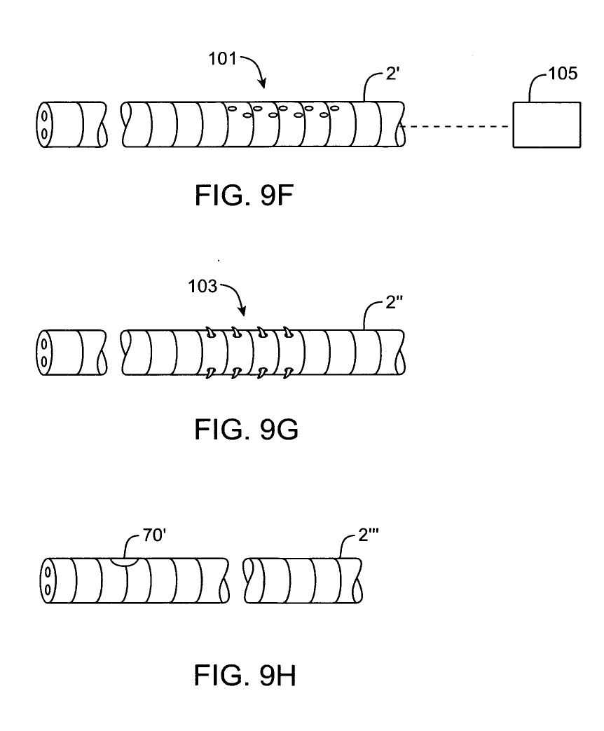

In yet another example of per-oral extra-gastric approaches to treating GERD, FIG. 9A shows an endoscopic body 2 which has been advanced through the stomach wall and through the diaphragm D for treating a hiatal hernia H. Endoscopic body 2 may be advanced through the gastric wall, through the diaphragm D, and then anchored to the diaphragm D in proximity to the hiatus HI and hiatal hernia HH in the same or similar manner as previously described. Once endoscopic body 2 has been desirably situated, endoscope 100 may be advanced into the thoracic cavity TC and articulated towards the hiatus HI, where needle assembly 114 may be utilized to deploy anchors 56, 58.

As shown in the top view in FIG. 9B of the hiatus HI and diaphragm D, anchors 56, 58 may be deployed within the diaphragm D about the periphery of hiatus HI. Anchors 56, 58 may be deployed on apposed sides of hiatus HI adjacent to esophagus E and hiatal hernia HH, or they may be deployed adjacent to hiatus HI. The stomach S may be drawn back through hiatus HI to alleviate the hiatal hernia HH through a number of methods. For instance, the endoscopic body 2 may be utilized to push or pull the stomach S back into the peritoneal cavity PC, or another tool, either laparoscopic or endoluminal, may be utilized to retract the hiatal hernia HH from the thoracic cavity TC back into the peritoneal cavity PC.

In either case, anchors 56, 58 may be deployed in the vicinity of hiatus HI and hiatal hernia HH such that when the anchors 56, 58 are drawn towards one another along suture 60, the region of the diaphragm D between and around the anchors 56, 58 are cinched and drawn close. This local cinching of the diaphragm is such that the opening of the hiatus HI is reduced to prevent the stomach S from slipping back through the hiatus HI and creating another hiatal hernia HH.

As mentioned above, stomach S may be drawn back through hiatus HI to alleviate the hiatal hernia HH through a number of methods. One example is shown in FIG. 9C where the tissue around or forming hiatal hernia HH may be adhered to endoscopic body 2 via a vacuum or suction force drawn through, e.g., opening 70. Once the tissue has been adhered, albeit temporarily, to endoscopic body 2, as shown by the arrows in FIG. 9D, endoscopic body 2 may be advanced distally through the esophagus E and into stomach S until hiatal hernia HH has been repositioned below the diaphragm D and within the peritoneal cavity PC, as illustrated in FIG. 9E. Once desirably repositioned, the tissue may be released from endoscopic body 2 and hiatus HI may be drawn into a smaller opening, as described and shown above in FIGS. 9A and 9B. Alternatively, the tissue of stomach S may then be affixed within the peritoneal cavity PC, e.g., via the diaphragm D as described below, or any of the procedures described herein may be carried out on the gastric tissue.

Aside from utilizing opening 70 for adhering tissue and drawing the adhered tissue through hiatus HI, other variations may include a plurality of openings 101 defined along a region of endoscopic body 2′, as shown in FIG. 9F. A vacuum source or pump 105 may be fluidly connected to endoscopic body 2′ to create the negative pressure for adhering the tissue thereto. Other variations may also include one or more retractable barbs or hooks 103, which may be projected and retracted from endoscopic body 2″ through respective openings, as shown in FIG. 9G. The retractable barbs or hooks 103 may likewise be located along a region of endoscopic body 2″. In yet another variation shown in FIG. 9H, endoscopic body 2′″ may utilize an opening 70′ located along body 2′″ near the distal end of the body 2′″. The proximity of opening 70′ near the distal end of the device may facilitate an ante-grade approach for endoscopic body 2′″ within the esophagus.

Although openings and barbs are described above for adhering the tissue thereto, these are merely illustrative of the variety of methods and devices which may be utilized to temporarily adhere the tissue to the endoscopic body and are not intended to be limiting in any way. Any number of other methods may also be utilized with the endoscopic body and are intended to be within the scope of this disclosure.

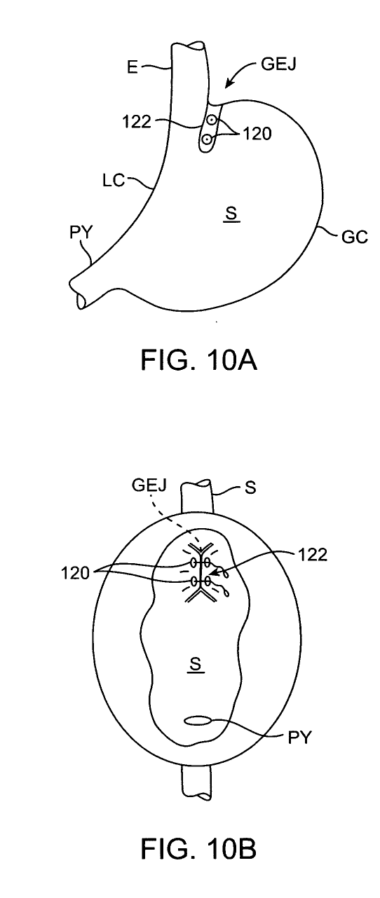

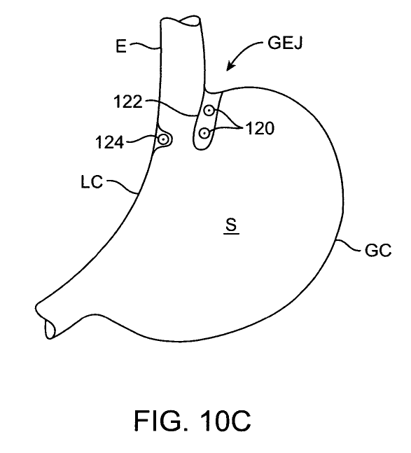

Utilizing any number of the intra-gastric or extra-gastric approaches described above (either alone or in combination with one another), a number of procedures may be accomplished for the treatment of GERD. FIG. 10A shows one variation in which the esophagus E may be lengthened by approximating a region of tissue along the anterior and posterior walls of stomach S inferiorly located and adjacent to the GEJ to create a small pouch, as also described above and in U.S. patent application Ser. Nos. 10/955,245 and 10/840,950, each of which have been incorporated herein by reference. Examples are also shown in U.S. patent application Ser. Nos. 10/735,030 filed Dec. 12, 2003 and in 11/002,575 filed Dec. 1, 2004, each of which is incorporated herein by reference in its entirety.

In creating a pouch with the tissue ridge 122 along the GEJ, two or more anchor pairs 120 may be deployed into the approximated tissue creating a ridge 122 which roughly follows the lesser curvature LC of stomach S. As few as one anchor pair 120 may be deployed into the tissue adjacent to the GEJ or three or even more pairs may be deployed into the tissue, as so desired. FIG. 10B shows stomach S in a partially open view when viewed from the side of the greater curvature GC towards the lesser curvature LC. As shown, the anchor pairs 120 (in this example two anchor pairs) may be seen extending from the GEJ towards the pylorus PY such that the GEJ opens directly behind tissue ridge 122.

In another example of a procedure, FIG. 10C shows a tissue configuration similar to the tissue ridge 122 of FIG. 10B but with an optional additional tissue fold 124 created along the lesser curvature LC in apposition to the distal end of tissue ridge 122. The resulting opening towards the esophagus E is additionally reduced and may further alleviate the symptoms of GERD.

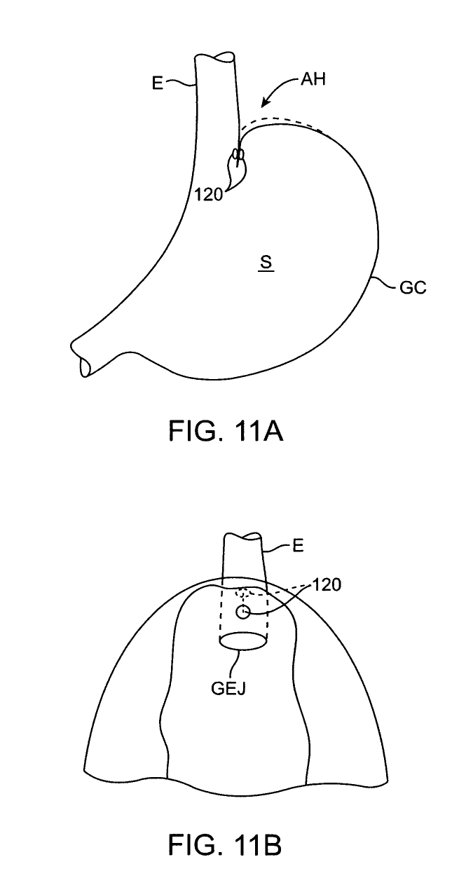

FIG. 11A shows an example in a side view of the stomach S with the angle of Hiss AH reconfigured by approximation of the esophageal wall E and the gastric wall resulting from any of the procedure as described above and shown in FIGS. 5E, 7B, or 8E utilizing either an intra-gastric or extra-gastric approach. As shown in the partially open stomach S viewed from the greater curvature GC in FIG. 11B, the anchor pair 120 may be seen extending from within the esophagus E into the stomach S.

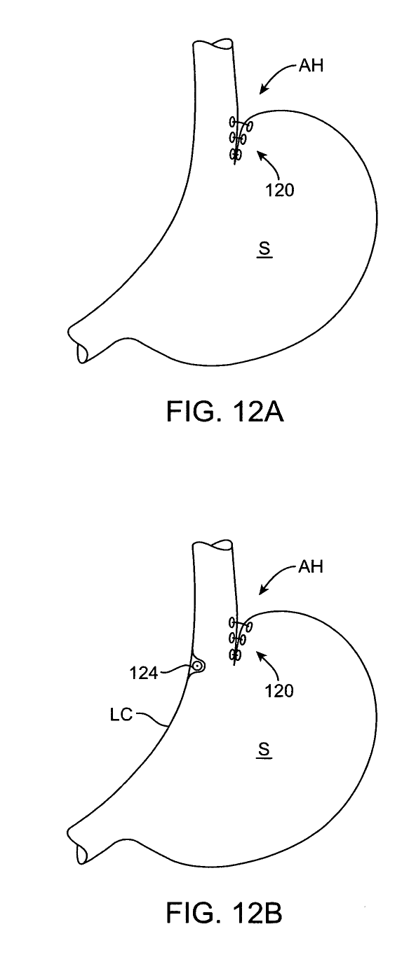

Another example similar to that shown in FIG. 11A is shown in the side view of FIG. 12A, where multiple anchor pairs 120 may be utilized to modify the angle of Hiss AH. In this example, the anchor pairs 120 may be deployed through any one of the above-described intra-gastric or extra-gastric approaches utilizing two, three, or more anchor pairs depending upon the desired degree of reconfiguration. Yet another example for modifying the angle of Hiss AH is shown in FIG. 12B, which not only reconfigures the angle of Hiss AH, as above, but also includes an additional tissue fold 124 created along the lesser curvature LC at, adjacent to, or inferior to the GEJ.

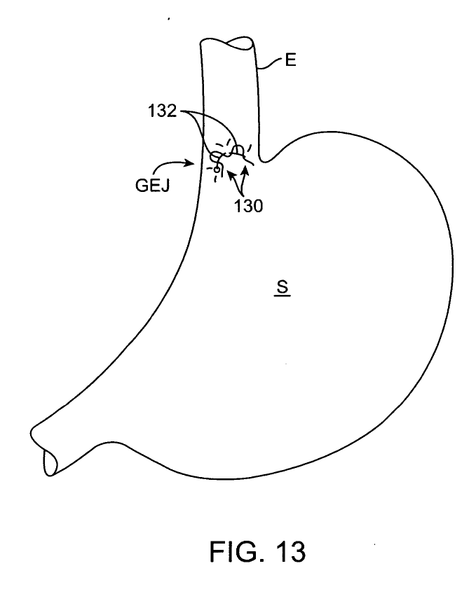

Aside from modifying the angle of Hiss, other procedures may be performed within or adjacent to the esophagus E. As shown in FIG. 13, one or more tissue folds 130 may be formed within or adjacent to the GEJ to help form a barrier to refluxing stomach contents. These tissue folds 130 may be approximated towards one another about the periphery of the esophagus via suture 132 to reduce the effective area of the GEJ.

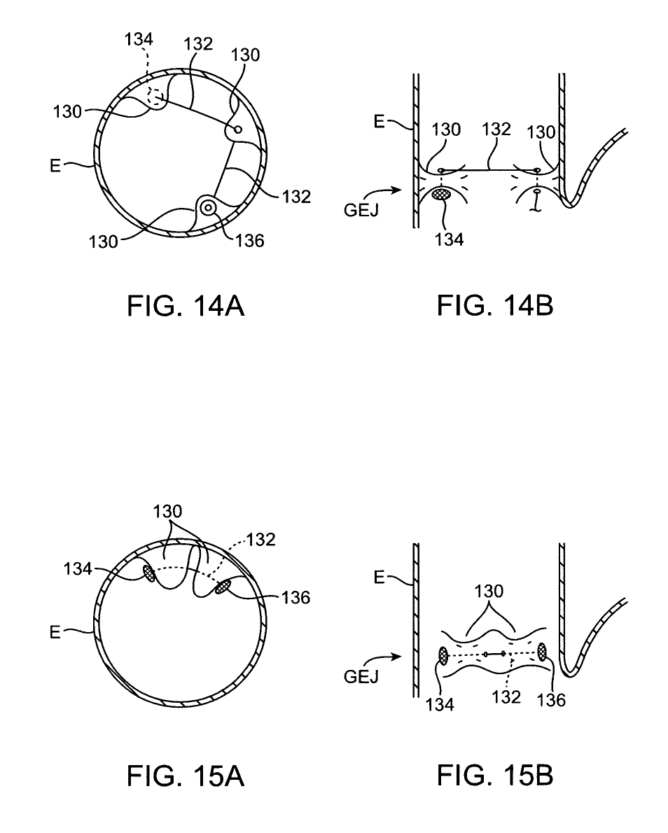

FIGS. 14A and 14B show top and side views, respectively, of the esophagus E with one or more tissue folds 130 formed in or around the GEJ utilizing soft tissue anchors 134, 136. Three tissue folds are shown formed about the periphery of esophagus E in this example but a single fold or more than three folds may alternatively be formed about the esophagus periphery. A first fold may be secured with first anchor 134 while the third fold may be secured with second anchor 136 such that the anchors are positioned atop, i.e., away from the stomach S, or below, i.e., towards the stomach S, their respective tissue folds such that the suture 132 passes through the tissue folds in a direction roughly parallel with the esophagus E. A length of suture 132 may be routed through all three tissue folds and connect the two anchors 134, 136 to one another resulting in a purse-string type approximation. The cinching of the anchors 134, 136 may serve to narrow the effective cross-sectional area of the esophagus and help to alleviate backflow of stomach contents into the esophagus E.

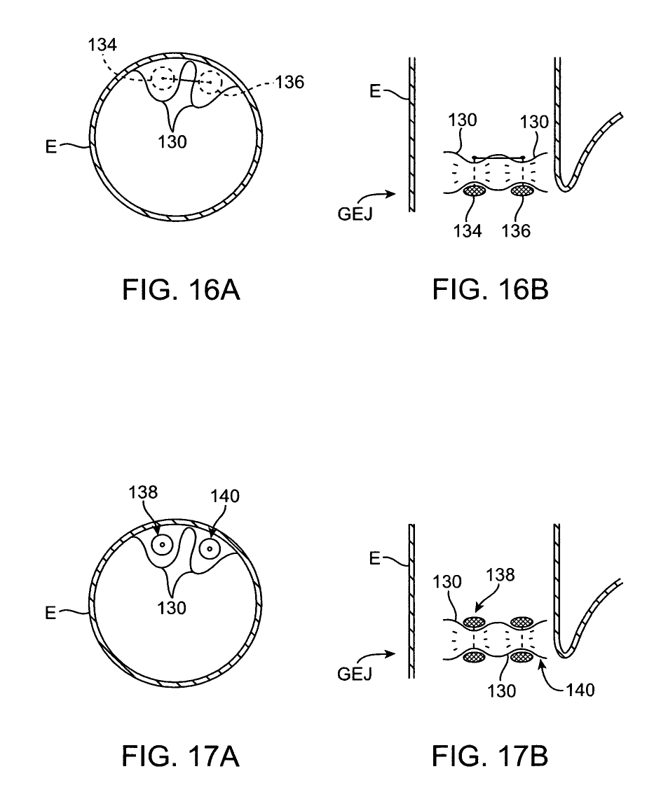

FIGS. 15A and 15B likewise show top and side views, respectively, of esophagus E with at least two tissue folds 130 approximated towards one another with first and second anchors 134, 136. The anchors 134, 136 in this example are placed relative to the tissue folds 130 such that the anchors 134, 136 are in apposition to one another. Although two tissue folds 130 are shown approximated in this example, a single tissue fold may be formed or three or more tissue folds may alternatively be formed approximated towards one another via the anchors 134, 136. FIGS. 16A and 16B also show top and side views, respectively, of esophagus E having two tissue folds 130 formed similarly to that in FIGS. 15A and 15B, but anchors 134, 136 may be positioned in this example either below the tissue folds, as shown, or atop the tissue folds and joined via the suture. FIGS. 17A and 17B show two tissue folds 130 which have been formed adjacent to one another with anchor pairs 138, 140 securing their respective tissue folds such that each individual fold has an anchor atop and below the fold for maintaining the tissue fold.

In each of the above examples, although specific numbers of tissue folds have been shown, this is intended to be merely illustrative and the number of tissue folds shown is not intended to be limiting in any way. As such, any number of tissue folds as practicable may be formed or approximated depending upon the desired results.

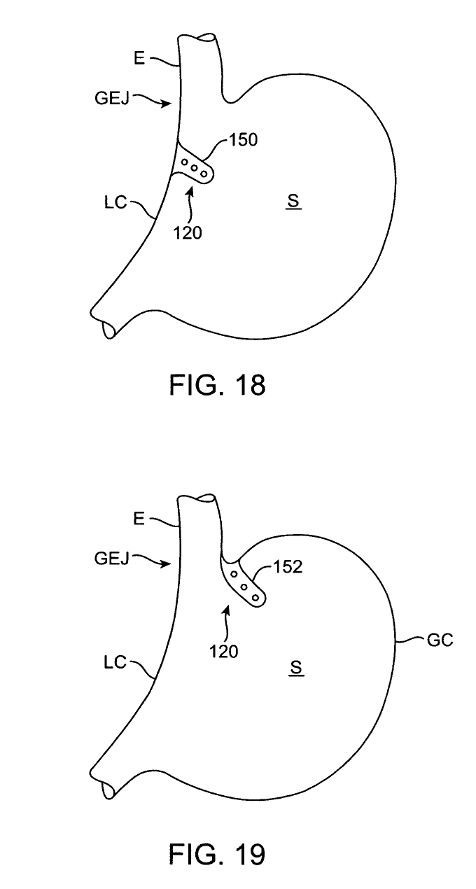

Other alternatives in forming tissue ridges within the stomach S may be seen in FIGS. 18 and 19. As shown in FIG. 18, a tissue ridge 150 may alternatively be formed below or inferior to the GEJ such that it extends from the lesser curvature LC and into the stomach cavity. Tissue ridge 150 may be formed utilizing any number of anchors 120 and it may be formed to extend from the lesser curvature LC at a variety of distances below the GEJ. Alternatively, tissue ridge 152 may also be formed adjacent to the GEJ such that it extends into the stomach S towards the greater curvature GC of the stomach S, as shown in FIG. 19.

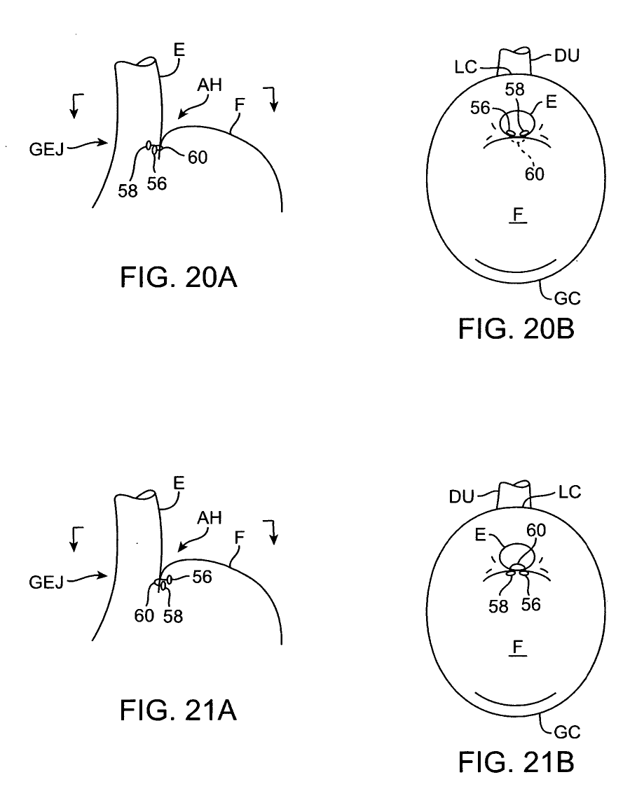

In yet another variation, tissue anchors 56, 58 may be utilized to approximate and secure the esophagus E to the stomach S such that the anchors 56, 58 are deployed adjacent to one another against the same side of the tissue and the suture 60 is formed into a U-stitch through the tissue layers. As shown in the side view of FIG. 20A and in the top view of stomach S of FIG. 20B, distal anchor 56 may be first deployed against the esophageal wall with the suture 60 pierced through the esophagus E and fundus F and then pierced back through the fundus F and esophagus E such that proximal anchor 58 may be also deployed within the esophagus E adjacent to distal anchor 56. The top view of FIG. 20B shows the cross-section of esophagus E, the top of fundus F, and a portion of the duodenum DU leading away from the stomach S for reference. FIGS. 21A and 21B likewise show the side view and top view, respectively, of the stomach S where a similar U-stitch may be formed within the tissue but with anchors 56, 58 having been deployed within the stomach S and with suture 60 passed through the fundus F, into the esophagus E, and then back through the esophagus E and through the fundus F. These procedures may be accomplished utilizing any one of the above-described intra-gastric or extra-gastric procedures.

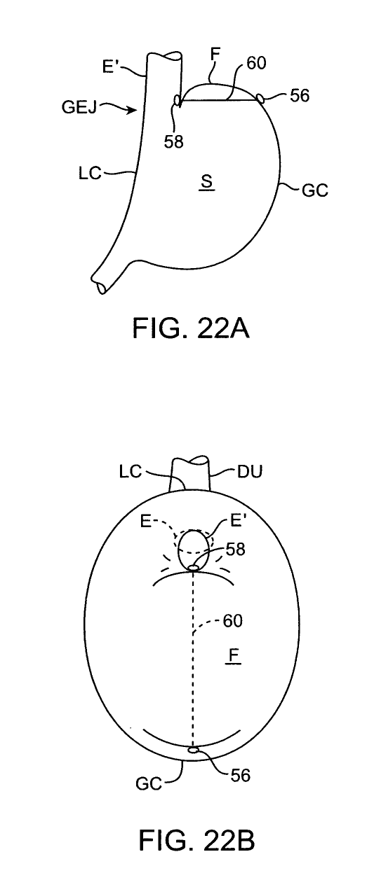

In yet another alternative shown in the side view of FIG. 22A, tissue anchors 56, 58 may be deployed within the esophagus E and on the outer surface of the stomach S along the greater curvature GC with suture 60 extending between the two. Cinching of the anchors 56, 58 towards one another may pull esophagus E such that it is narrowed bringing apposed sides of the esophagus E′ towards one another, as shown in the top view of stomach S in FIG. 22B. Anchor 56 may be positioned along the greater curvature GC or any other location along the outer serosal surface of the stomach S provided that the suture 60, when tensioned, may draw the cross-sectional area of the esophagus E into the narrowed esophagus E′. The anchors 56, 58 may be deployed utilizing any of the intra-gastric or extra-gastric approaches described above.

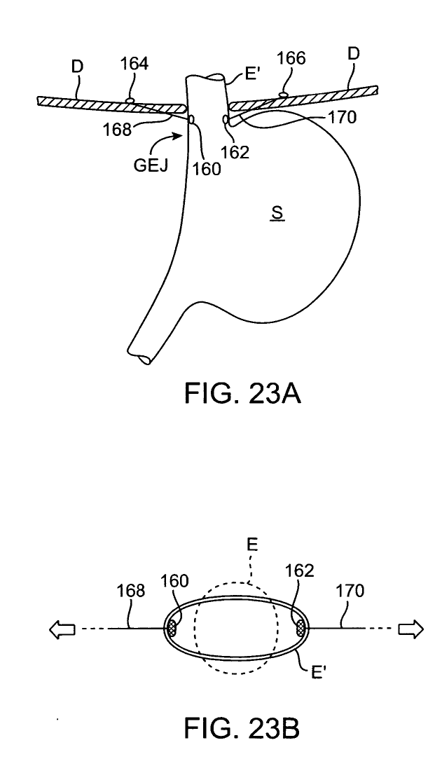

In another alternative shown in FIG. 23A, the esophagus E may be narrowed into the narrowed esophagus E′ by deploying first anchor 160 within the esophagus and second anchor 164 within or atop the diaphragm D with the two anchors connected via suture 168. Another first anchor 162 may also be deployed within the esophagus E at a location opposite to where anchor 160 is positioned and another second anchor 166 may be deployed within or atop the diaphragm D with the anchors 162, 166 connected via suture 170. Anchor 166 may be deployed at a location opposite to where anchor 164 is located on diaphragm D. When anchors 160, 164 are drawn towards one another and secured, and when anchors 162, 166 are likewise drawn towards one another and secured, the resulting cross-section of esophagus E becomes narrowed into esophagus E′, as shown in the top view of esophagus E, E′ in FIG. 23B.

Although a number of illustrative variations are described above, it will be apparent to those skilled in the art that various changes and modifications may be made thereto without departing from the scope of the invention. Moreover, although specific configurations and applications may be shown, it is intended that the various features may be utilized in various combinations and in various types of procedures as practicable. It is intended in the appended claims to cover all such changes and modifications that fall within the true spirit and scope of the invention.

1. A method for treating gastroesophageal reflux disease, comprising:
advancing trans-esophageally an endoscopic body adapted to shape-lock a selected configuration; and
modifying tissue in or around a gastroesophageal junction with at least one tool disposed at a distal end of the endoscopic body.

2. The method of claim 1 further comprising locking the selected configuration of the endoscopic body within the esophagus after advancing trans-esophageally.

3. The method of claim 1 wherein advancing trans-esophageally comprises advancing the endoscopic body in a flexible state.

4. The method of claim 1 wherein advancing trans-esophageally comprises advancing the distal end of the endoscopic body within a proximity of the gastroesophageal junction.

5. The method of claim 4 wherein advancing the distal end of the endoscopic body comprises advancing the distal end to a position superior to the gastroesophageal junction.

6. The method of claim 4 wherein advancing the distal end of the endoscopic body comprises advancing the distal end to a position adjacent to the gastroesophageal junction such that a distal portion of the endoscopic body is retroflexed within a stomach.

7. The method of claim 1 wherein advancing trans-esophageally comprises securing a distal end of the endoscopic body to an opening in a stomach wall.

8. The method of claim 7 further comprising advancing an endoscope through the endoscopic body into a peritoneal or thoracic cavity within a patient.

9. The method of claim 1 wherein modifying tissue comprises distally advancing a needle assembly from the endoscopic body into the tissue in or around the gastroesophageal junction.

10. The method of claim 9 further comprising deploying at least two expandable anchors slidingly interconnected via suture from the needle assembly against the tissue.

11. The method of claim 10 further comprising approximating the at least two expandable anchors over the suture such that an outer surface of esophageal tissue is secured against an outer surface of stomach tissue.

12. The method of claim 10 wherein deploying at least two expandable anchors comprises deploying the anchors within the esophagus adjacent to one another such that the suture forms a U-stitch relative to the gastroesophageal junction.

13. The method of claim 10 wherein deploying at least two expandable anchors comprises deploying the anchors within a stomach adjacent to one another such that the suture forms a U-stitch relative to the gastroesophageal junction.

14. The method of claim 1 further comprising adhering the tissue in or around the gastroesophageal junction to a region of the endoscopic body and advancing the adhered tissue distally past a diaphragm of a patient prior to modifying tissue.

15. The method of claim 14 further comprising securing the tissue below the diaphragm of the patient.

16. The method of claim 14 wherein adhering the tissue comprises adhering the tissue via a vacuum force.

17. The method of claim 14 wherein adhering the tissue comprises adhering the tissue via one or more retractable hooks or barbs projecting from a surface of the endoscopic body.

18. A method for treating gastroesophageal reflux disease, comprising:
advancing trans-esophageally an endoscopic body adapted to shape-lock a selected configuration into a stomach;
positioning the endoscopic body adjacent a tissue region of interest within the stomach;
locking the selected configuration of the endoscopic body; and
approximating tissue from the tissue region of interest such that at least one tissue fold is formed within the stomach.

19. The method of claim 18 wherein advancing trans-esophageally comprises advancing the endoscopic body in a flexible state.

20. The method of claim 18 wherein approximating tissue comprises approximating tissue from an anterior region and a posterior region of the stomach such that a tissue pouch is formed.

21. The method of claim 20 wherein the tissue pouch extends from a gastroesophageal junction into the stomach.

22. The method of claim 18 further comprising securing the approximated tissue.

23. The method of claim 22 wherein securing comprises deploying at least one pair of expandable anchors.

24. The method of claim 18 wherein advancing trans-esophageally comprises adhering stomach tissue located superior to a hiatus opening to a region of the endoscopic body and advancing the adhered tissue distally past the hiatus opening.

25. The method of claim 24 wherein adhering stomach tissue comprises adhering the tissue via a vacuum force.

26. The method of claim 24 wherein adhering stomach tissue comprises adhering the tissue via one or more retractable hooks or barbs projecting from a surface of the endoscopic body.

27. A method for treating gastroesophageal reflux disease, comprising:
advancing trans-esophageally an endoscopic body adapted to shape-lock a selected configuration; and
forming at least one tissue fold in or around a gastroesophageal junction with at least one tool disposed at a distal end of the endoscopic body.

28. The method of claim 27 further comprising locking the selected configuration of the endoscopic body within the esophagus after advancing trans-esophageally.

29. The method of claim 27 wherein advancing trans-esophageally comprises advancing the endoscopic body in a flexible state.

30. The method of claim 27 wherein forming at least one tissue fold comprises deploying at least one pair of expandable tissue anchors within or against the tissue fold.

31. The method of claim 27 wherein forming at least one tissue fold comprises forming the at least one tissue fold such that a cross-sectional area of the esophagus is reduced.

32. The method of claim 27 wherein forming at least one tissue fold comprises forming at least one additional adjacent tissue fold.

33. The method of claim 32 wherein forming at least one additional adjacent tissue fold comprises forming the additional tissue fold such that both tissue folds are connected to one another via suture.

34. The method of claim 27 wherein advancing trans-esophageally comprises adhering stomach tissue located superior to a hiatus opening to a region of the endoscopic body and advancing the adhered tissue distally past the hiatus opening.
